(19) United States
(12) Patent Application Publication
(10) Pub. No.: US 2004/0062694 A1
(43) Pub. Date: Apr. 1, 2004

(54) One-piece connector for assembling a sterile medical product

(76) Inventors: Mark R. Vandlik, Mundelein, IL (US); Vick M. Crawley, Mountain Home, AR (US)

Correspondence Address: Bradford R. L. Price, Baxter Healthcare Corporation, Fenwal Division, RLP-30, P.O. Box 490 - Route 120 & Wilson Road, Round Lake, IL, 60073, US

(21) Appl. No.: 10/262,012

(22) Filed: Oct. 1, 2002

(51) Int. Cl. A61L 9/00; A62B 7/08; A61L 2/00; A61L 2/08

(52) U.S. Cl. 422/305; 422/306; 422/307; 422/121; 422/22; 422/26

(57) ABSTRACT

A connector for joining two parts of a medical product, where the parts are sterilized by different forms of sterilization, is disclosed. The connector includes a generally tubular body having a first end adapted for connection to a part of the medical product, another closed, but openable end, and an expansion chamber of one-piece integral construction with the tubular body.

The present invention generally relates to medical products and methods for assembling and sterilizing such products. More particularly, the present invention relates to a one-piece connector for joining one part of a disposable medical product, which has been sterilized by a first form of sterilization, to another part of the medical product sterilized by a different form of sterilization.

### BACKGROUND OF THE INVENTION

Medical products are routinely sterilized prior to use. There are several different forms of sterilization which may be used to sterilize medical products, some forms being better suited for certain products or parts of a product than other forms. For example, radiation sterilization, such as electron beam radiation or gamma radiation, is effective and often preferred for sterilizing plastic tubes and other plastic parts of a medical product. Gas sterilization, such as by ethylene oxide (EtO), may also be effective in sterilizing plastic parts that are open to access by the gas.

However, where the medical product includes a liquid component sealed within a plastic container, radiation sterilization and gas sterilization may not be suitable. Gas sterilization would be ineffective to sterilize the sealed liquid, and exposing the liquid to radiation may lead to product degradation or otherwise have a deleterious effect on the liquid. For sterilizing a liquid sealed in a container, the selected form of sterilization may be steam sterilization, also known as autoclaving. Steam sterilization is less well suited for sterilizing “dry” parts.

Some medical products, such as the disposable fluid circuits used in blood donation or collection, include both a liquid component and a non-liquid component. For example, the disposable fluid circuits may include one or more containers containing a liquid that is pre-attached to a network of empty plastic containers, tubing and other plastic parts (e.g., separation devices, valving and pumping cassettes). For the reasons described above, such medical products cannot be sterilized by a single form of sterilization. Thus, in these cases, the component parts of the product must be separately sterilized and the product assembled to provide a finished sterile product. Additional sterilization of the product at the point of attachment of the component parts may also be required.

U.S. Pat. Nos. 5,496,302 and 5,009,654, assigned to the assignee of the present application and hereby incorporated herein by reference, describe methods and systems for assembling a medical product where the medical product is made up of portions sterilized by different forms of sterilization. The methods described therein generally involve bringing together the ends of plastic tubes associated with the respective parts of the medical product, isolating the flow paths within the plastic tubes to prevent the ingress of bacteria into the parts of the medical product, joining the ends of the tubing of the medical product and sterilizing at least the connection site with a selected form of sterilization while shielding the remainder of the product from the form of sterilization.

In the system described in the aforementioned patents, the part of the product that includes the liquid containers (i.e., “the wet side”) is sterilized by steam sterilization. The remaining, non-liquid part of the product (i.e., “the dry side”) is sterilized by a different form of sterilization, such as gas or radiation sterilization. Each part of the product includes one or more tubing segments associated with the product part.

In addition, at least the “wet side” of the product (i.e., the plastic container(s) with associated tubing) includes an openable barrier to flow, such as a frangible closure device, in the tubing flow path. (The barrier is opened at the appropriate time by the end user.) Presently, the frangible is housed inside a first bushing, one end of which is joined to a tubular access port on the container. The other end of the first bushing is, in turn, joined to one end of a tubing segment that is eventually joined to tubing from the “dry side.” The opposite end of the tubing segment is sealed with a separately provided air-filled pillow that protects the tubing segment from collapse during autoclaving. A liquid component or “wet side” of a disposable fluid circuit is shown in FIG. 2.

The final product is assembled by joining the tubing ends from the “wet” and “dry” sides, which typically have been pre-sterilized by their respective forms of sterilization. To preserve the sterility of most of the product parts, the flow paths must be isolated from the remainder of the respective product parts. Thus, the tubing segments are squeezed by clamps or other means to isolate the exposed ends of the product portions from their respective remainders. Once the flow paths have been isolated, the sealed ends are opened and the two parts of the product can be joined. A second bushing is provided and acts as an adapter for the two tubing ends. The tubing ends are inserted into the second bushing and bonded thereto. Finally, the connection site up to the points of isolation must then be re-sterilized to provide the finished, sterile product.

Although, the methods and systems described above have provided a significant improvement over previous systems, it will be appreciated that several assembly, isolation and sterilization steps are required to arrive at the finished, sterile product. Thus, efforts continue to further streamline the assembly and sterilization of disposable medical products by, for example, reducing the required number of assembly and sterilization steps. These efforts have resulted in the present invention, whereby the step of separately isolating the flow paths (with clamps) is no longer required. In addition, separately providing and bonding first and second bushings and air pillows to a long strand of tubing is also no longer required. Thus, as a result of the present invention, a savings in the cost of materials and assembly can be attained.

### SUMMARY OF THE INVENTION

In one aspect, the present invention is directed to a one-piece connector for providing flow communication between two medical products. The one-piece connector may include an elongated, generally tubular body having a first end adapted for connection to a medical product and a second closed end. The one-piece connector further includes an expansion chamber of one-piece integral construction with the tubular body. The expansion chamber protects the tubular body of the connector from collapse during autoclaving. The second closed end, when opened provides an instant connection site for receiving the tubing of the other product or part thereof.

In another aspect, the present invention is directed to a medical product sterilized by a first form of sterilization. The medical product includes a tubular connection site that includes a one-piece connector comprising an elongated, generally tubular body having a first end connected to the medical product and a second closed end. The connection site and, more specifically, the connector includes an expansion chamber of one-piece integral construction with the tubular body located at the sealed second end.

### BRIEF DESCRIPTION OF THE DRAWINGS

FIG. 1 is a perspective view of an exemplary blood processing system that includes a hardware unit and a disposable fluid circuit including parts that are sterilized by different forms of sterilization;

FIG. 2 is a plan view of a prior art “wet side” portion of a disposable fluid circuit;

FIG. 3 is a partial plan view of an assembled medical product that includes a portion that is sterilized by one form of sterilization and is attached, by connectors embodying the present invention, to containers holding a medical fluid;

FIG. 4 is an enlarged cross-sectional view of the connector embodying the present invention.

FIG. 5 is a plan view of a medical liquid container with an attached connector embodying the present invention;

FIG. 6 is a side view of the container and connector of FIG. 5;

FIG. 7 is an enlarged cross-sectional view of the connector tubular body;

FIG. 8 is a diagrammatic plan view of an assembled medical product being subjected to radiation sterilization while shielding at least a portion of the medical product from the radiation; and

FIG. 9 is a end view of the portion of a plastic tube inserted within the connector embodying the present invention.

### DETAILED DESCRIPTION OF THE EMBODIMENTS

The present invention will be described in the context of one of its preferred uses, namely, as a connector for use with a medical product such as a disposable fluid circuit used to process blood and/or other biological fluids. However, it will be understood that the present invention is not limited to use with the product described below, or to systems used for processing blood, or even to products used in the medical field. The connector of the present invention may be used in any setting where component parts of a product are sterilized by different forms of sterilization and joined to provide an integral sterile product.

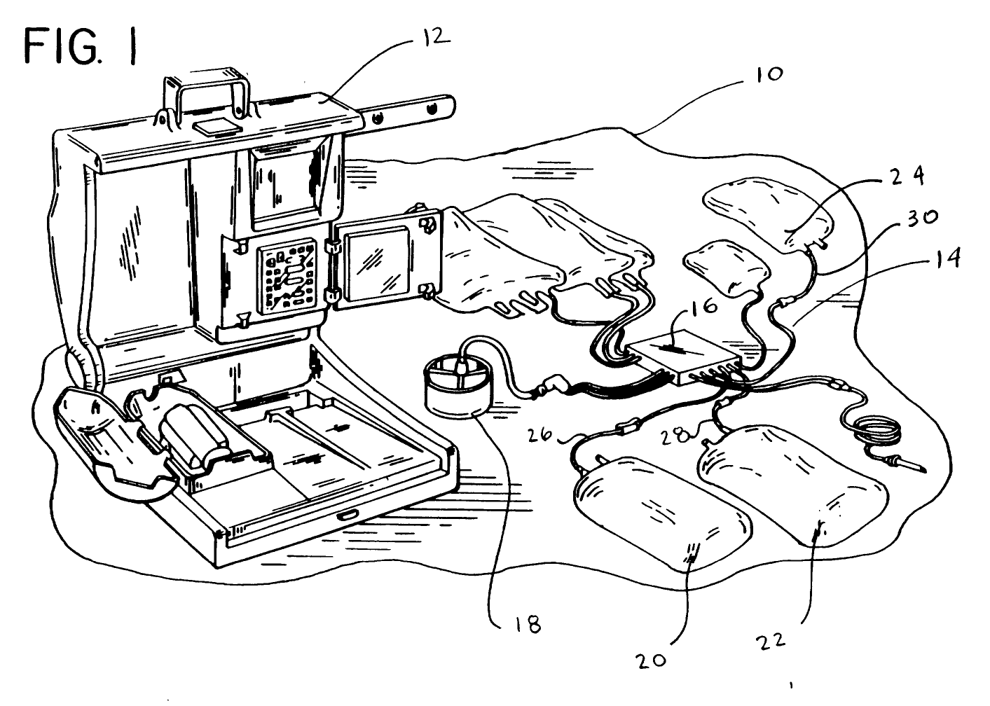

Turning now to the drawings, FIG. 1 shows, for illustrative purposes only, a system 10 used to collect blood from a donor and separate blood into its components (e.g., red blood cells, platelets, plasma). The details of the blood processing system shown in FIG. 1 are described in U.S. Pat. No. 6,348,156, assigned to the assignee of the present application and incorporated herein by reference. Other fluid circuits for use with other blood or fluid processing devices may also utilize the connector of the present invention. Such fluid circuits are described in U.S. Pat. Nos. 5,868,696 and 5,135,667, also incorporated herein by reference.

As shown in FIG. 1, the system 10 includes a hardware unit 12 and a disposable fluid circuit 14 for mounting onto hardware unit 12. As shown in FIG. 1, fluid circuit 14 includes several different components joined together. For example, in the illustrated system, fluid circuit 14 may include a cassette 16 attached by tubing to a separation device 18 and multiple containers. Plastic containers 20, 22 and 24, hold liquids used in the blood separation procedure (e.g., anticoagulant, saline, preservative solutions and the like). The part of the product including the liquid containers is commonly referred to as “the wet side.” The remaining (i.e., non-liquid) part of the product is commonly referred to as “the dry side.” The fluid circuit 14 is provided to the end user as a fully-assembled, pre-sterilized disposable kit that includes both the “wet” and “dry” sides.

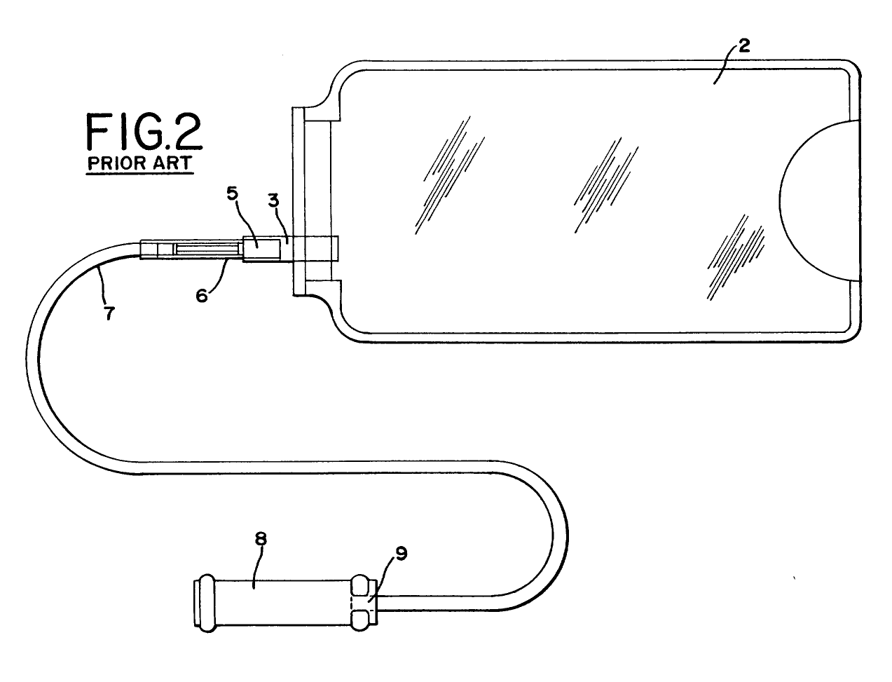

As described above, the “wet” and “dry” sides are not amenable to sterilization by a single form of sterilization. The multiple steps required to assemble and sterilize, (and resterilize) such products have also been previously described. For example, with reference to FIG. 2, providing the liquid containers(s) 2 of the “wet side” involves inserting a frangible closure device 5 into a first bushing 6, and bonding one end of the bushing to a port 3 on the container 2. A tubing segment 7 (which is later attached to tubing from the “dry side”) is then attached to the other end of bushing 6. The free end of tubing segment 7 is sealed with pillow 8. The assembled wet side is then sterilized, typically by steam sterilization, the flow path is isolated, the end 9 is exposed and joined to the “dry side,” as previously described. As set forth in more detail below, with the present invention, many of these steps have now been eliminated.

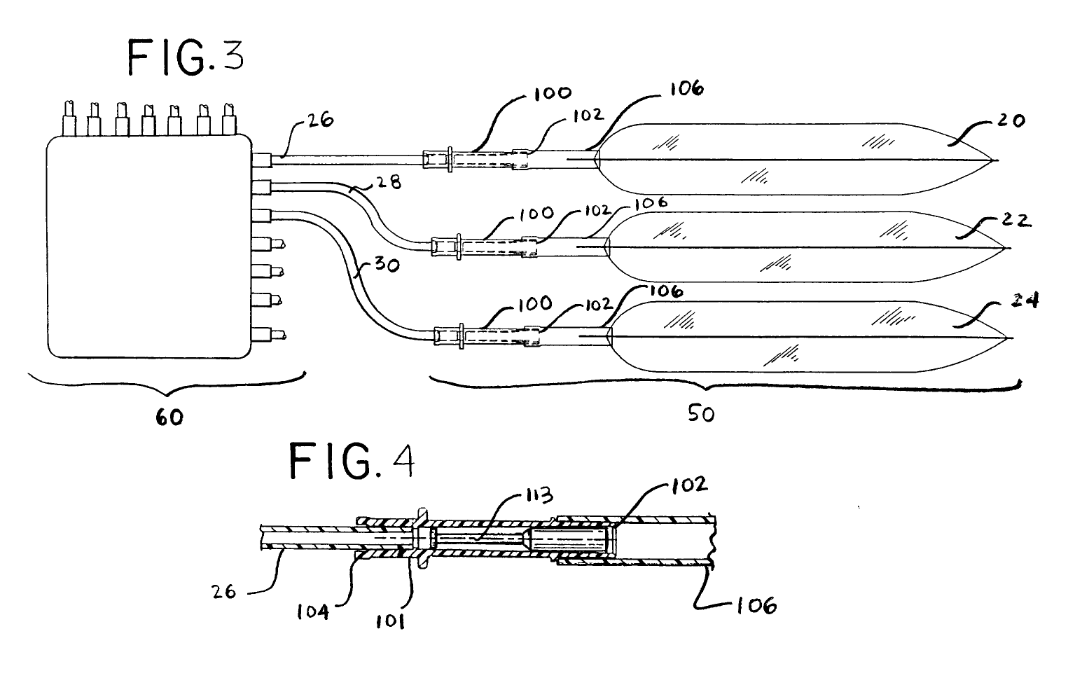

Shown in FIG. 3 is a partial view of a disposable fluid circuit of the type shown in FIG. 1. The fluid circuit includes a “wet side” 50 that includes a plurality of containers 20, 22 and 24 containing a liquid (i.e., the “wet side”). The fluid circuit also includes a “dry side” 60, represented as cassette 16 with plastic tubes 26, 28 and 30 extending therefrom. The “wet” and “dry” sides are joined by connector 100 embodying the present invention.

In one embodiment, connector 100 may be provided as an elongated member having a generally tubular body or housing 101 with a first end 102 and a second end 104. First end 102 is open and is adapted to be joined to a tubing port 106 of a container (20, 22 or 24). Second end 104 is closed but, as described below, can be “opened” to receive tubing (26, 28 and/or 30) from the “dry side” of the product 12.

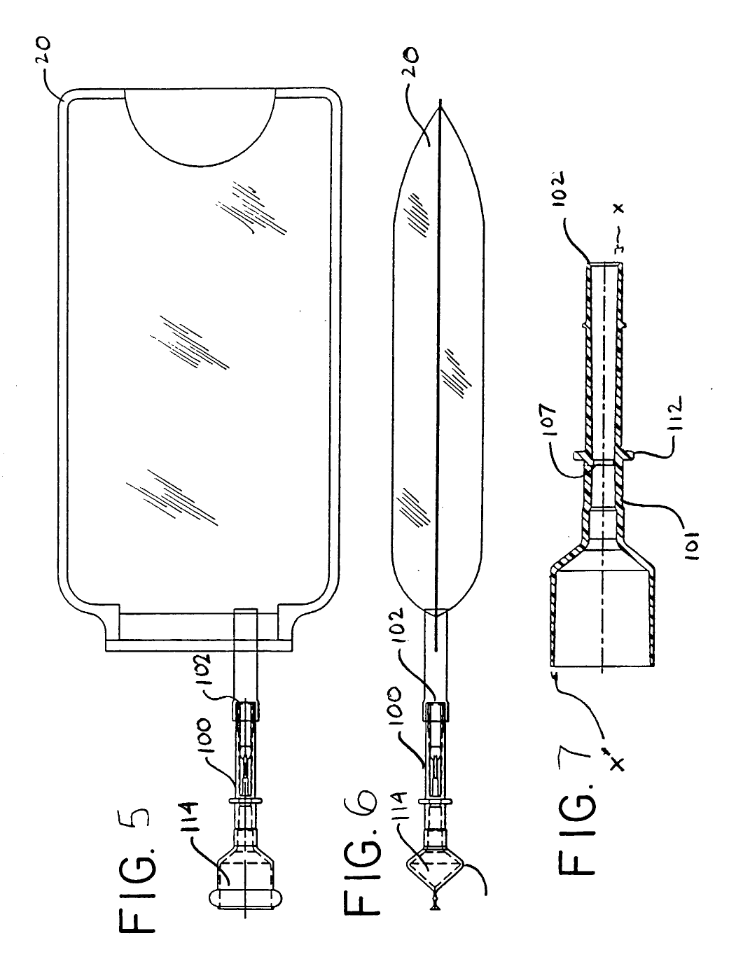

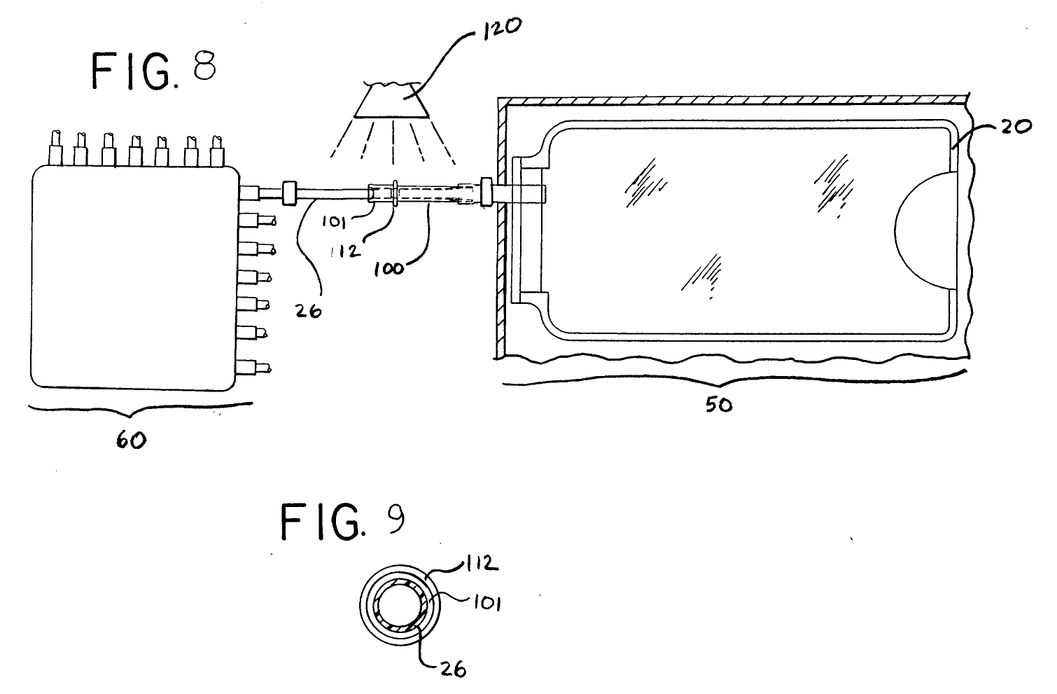

As shown in FIGS. 5 and 6, second end 104 of connector 100 terminates in an air-filled expansion chamber or pillow 114. Expansion chamber 114, which is integral with tubular body 110 of connector 100, is a portion of tubular body 101 having an enlarged cross-section. The wall of tubular body 101 in the vicinity of second end 104 (i.e., at expansion chamber 114) is reduced in thickness as compared to the remainder of tubular body. For example, as shown in FIG. 7, where the thickness of the wall, identified by the letter “x,” at or near first end 102 may be approximately 0.05 inches, the thickness of wall at second end 104 identified by x′ may be approximately 0.025 inches. As will be recognized by those of skill in the art, the reduced thickness of the tubular wall of expansion chamber 114 ensures that the increased pressure of the autoclaving process will not cause the remainder of tubular body 101 to collapse. The air-filled expansion chamber 114 is provided by sealing second end 104 of tubular body 101, as shown in FIG. 6. A preferred means of sealing second end 104 of tubular body 101 is radio-frequency (RF) sealing.

As shown in FIG. 7, when opened, second end 104 may be funnel-shaped to provide a lead-in for the tube of the “dry side,” allowing for easy insertion of the tube. As also shown in FIG. 7, the inner wall of tubular body includes a rim 107 near flange 112 (described below). Rim 107 acts as a stop for tubing (26, 28 or 30) inserted into second end 104. Tubular body 101 includes ring or flange 112 located between first end 102 and second end 104. Flange 112 serves as a guide for positioning connector 100 in a holder during assembly and sterilization of the final product.

Referring back briefly to FIG. 4, the internal chamber of tubular body 101 includes an openable barrier 113 which prevents flow communication between the “wet side” and the “dry side” of the medical product. However, barrier is openable (by the end user) to allow flow at the appropriate time. In one embodiment, barrier may be a frangible connector, such as those described in U.S. Pat. Nos. 4,181,140 and 4,294,247, incorporated herein by reference.

Tubular body 101 of connector 100 may be made of any biocompatible plastic material that can be steam sterilized and exposed to radiation sterilization without damaging the connector 100. One such preferred material for connector 100 is polyvinyl chloride plasticized with a plasticizer. Polyvinyl chloride is used in many medical applications and is sterilized by autoclaving. Other suitable materials may include other polyolefins, which can be subjected to steam sterilization and exposed to radiation sterilization. Connector 100 is preferably a one-piece integral connector that is preferably injection molded. Alternatively, connector 100 may be made by bubble tubing extrusion.

Containers of the “wet side” may be provided with the connector 100 pre-attached. Specifically, connector 100 may be attached to port 106 (FIGS. 3-4) of the container by known techniques. A preferred technique is solvent bonding. Connector 100 and the container are then pre-sterilized, such as by autoclaving. The other part of the medical product (the “dry side”) will typically not be pre-sterilized. The “dry side” of the medical product is sterilized after joinder and is sterilized by, preferably, radiation sterilization. Preferred forms of radiation sterilization include electron beam radiation and gamma radiation.

The sterilized “wet side” is brought together with the non-sterilized components of the “dry side.” The end of the tube(s) 26, 28, 30 is brought into close proximity with the sealed end 104 of connector 100. Sealed second end 104 is then opened by tearing or cutting expansion chamber 114 to expose second end 104. Once second end 104 has been opened, tubing (26, 28 or 30) from the “dry side” is inserted into open second end 104, as generally shown in FIG. 4. The tubing end of “dry side” 60 is bonded to open end 104, preferably, by solvent bonding.

With the entire product now assembled, the non-sterilized part of the product and at least part of the connection site is ready to be sterilized. As discussed previously, the preferred form of sterilization is radiation sterilization and, more preferably, electron beam irradiation. The non-sterilized part of the product and a part of connector 100 are positioned within the field of an electron beam (provided by an electron beam accelerator 120 shown generally in FIG. 8) to expose at least the “dry side” of the product, and at least a portion of connector 100 to an electron beam. Details of the dosage and time required to effectively sterilize the assembled medical product are beyond the scope of the present invention. However, a typical dose of electron beam energy required to sterilize the entire “dry side” of the assembled product would be approximately 10 MeV. During sterilization of the “dry side” and connector 100, containers (20, 22 and 24) are shielded from the electron beam radiation to avoid degradation of the product contained therein.

Connector 100 provides a one-piece unit that has built-in: an access site for attachment of the “dry side;” an openable flow barrier; and an expansion chamber or pillow to protect against tubing collapse during autoclaving. With connector 100, several parts and steps previously required can be eliminated. For example, a separate bushing for joining the tubing ends from the respective portions of the product parts is no longer required, as connector 100 provides an access site for receiving the tubing end from the dry side. A separately attached expansion chamber is also no longer required, as connector 100 includes an integral pillow 114. Further advantages of connector 100 may also be apparent to those of skill in the art.

Although the present invention has been described in terms of the preferred embodiment for use with a specific product as an example of how it may be employed, the present invention is not limited to the same. The scope of the present invention is defined by the appended claims.

What is claimed:

1. A one-piece connector for providing flow communication between two medical products comprising:
a generally tubular body having a first end adapted for connection to a medical product and a second closed end; and
an expansion chamber of one-piece integral construction with said tubular body.

2. The connector of claim 1 wherein said connector is injection molded.

3. The connector of claim 1 wherein said connector is formed by bubble tubing extrusion.

4. The connector of claim 1 wherein said connector is made of a flexible plastic material.

5. The connector of claim 4 wherein said flexible plastic material comprises polyvinyl chloride.

6. The connector of claim 1 wherein said expansion chamber is located at said second end.

7. The connector of claim 6 wherein said expansion chamber comprises an enlarged section of said tubular body.

8. The connector of claim 1 wherein said tubular body has a wall thickness that is greater at said first end than at said second end.

9. The connector of claim 1 wherein said second end is openable by cutting or tearing said expansion chamber.

10. The connector of claim 1 wherein said connector is exposable to radiation sterilization and steam sterilization without being adversely affected by said forms of sterilization.

11. A medical product sterilized by a first form of sterilization, said product comprising a tubular connection site comprising:
a one-piece connector comprising a generally tubular body having a first end connected to said medical product and a second closed end; and
an expansion chamber of one-piece integral construction with said tubular body located at said second end.

12. The medical product of claim 11 comprising a plastic container and a medical fluid contained therein.

13. The medical product of claim 11 wherein said medical product is sterilized by steam sterilization.

14. The medical product of claim 11 wherein said second end is openable.

15. The medical product of claim 14 wherein said second end is openable by cutting or tearing said expansion chamber.

16. The medical product of claim 14 wherein after opening said second end, said open second end is adapted to receive at least a portion of a second medical product.

17. The medical product of claim 11 wherein said first form of sterilization is steam sterilization and said connector is exposable to radiation sterilization without being adversely affected by said forms of sterilization.

18. The medical product of claim 16 wherein said second end is adapted to receive a plastic tube defining a flow path to said second medical product.

19. The medical product of claim 11 wherein said connector includes a frangible barrier between said first and second ends of said tubular body.

20. The medical product of claim 11 wherein said expansion chamber is substantially filled with air.

21. The medical product of claim 11 comprising an access port, wherein said connector is attached to said access port.
